(19) United States
(12) Patent Application Publication
(10) Pub. No.: US 2026/0287582 A1
(43) Pub. Date: Sep. 24, 2026

(54) DELIVERY AND MEASUREMENT OF FLUORESCENT NANOCRYSTALS IN BIOLOGICAL TISSUE

(71) Applicants: Rijksuniversiteit Groningen, Groningen (NL); Academisch Ziekenhuis Groningen, Groningen (NL)

(72) Inventors: Alina SIGAEVA, Hässelby (SE); Siyu FAN, Shanghai (CN); Yue ZHANG, Mainz (DE); Arturo ELÍAS LLUMBET, Groningen (NL); Maria Margarida DIAS CARMONA LOBITA, Groningen (NL); Mohammad-Ali SHAHBAZI, Meerstad (NL); Hélder ALMEIDA SANTOS, Groningen (NL); Romana SCHIRHAGL, Groningen (NL)

(21) Appl. No.: 19/489,305

(22) PCT Filed: Jun. 4, 2024
(86) PCT No.: PCT/NL2024/050284

§ 371 Date: Dec. 2, 2025

(30) Foreign Application Priority Data
Jun. 5, 2023 (EP) 23177366.4

(51) Int. Cl. G01N 33/543; G01N 21/64; G01N 33/533

(52) U.S. Cl. CPC G01N 33/54346; G01N 21/6428; G01N 33/533

(57) ABSTRACT

A microneedle assembly (10) for delivering fluorescent nanocrystals (FNC) into a tissue (T) comprises one or more microneedles (11). Each microneedle (11) comprises a plurality of fluorescent nanocrystals (FNC) dispersed throughout a solid matrix material (11m). The tissue (T) can be penetrated with the one or more microneedles (11) and at least part of the matrix material (11m) can be dissolved inside the tissue for delivering the fluorescent nanocrystals (FNC) into the tissue (T). Various aspects of the tissue (T) can be measured by delivering light pulses to the tissue (T), and measuring a resultant fluorescent signal (Sf) from the fluorescent nanocrystals (FNC) inside the tissue (T). The microneedle assembly (10) can be, for example, manufactured using micro-molding techniques using a liquid precursor of the matrix material (11m) with a concentration of the fluorescent nanocrystals (FNC) in the liquid precursor.

### TECHNICAL FIELD AND BACKGROUND

The present disclosure relates to methods, systems, and devices for delivering fluorescent nanocrystals (FNC) into a tissue and/or performing measurements in a tissue with FNC. The disclosure further relates to a microneedle assembly and methods for manufacturing such assembly.

In biology, radicals such as reactive oxygen species, play a crucial role. Free radicals are chemically reactive molecules containing an unpaired electron in their outer shell, making them highly reactive and potentially damaging to biological molecules. While high levels of free radicals can be harmful and lead to oxidative stress, moderate levels of free radicals have important physiological functions in various biological processes. For example, psychological stress can trigger the production of free radicals, leading to oxidative stress. Chronic oxidative stress from increased free radicals can damage cells and contribute to diseases. Detecting radicals can be difficult, e.g., due to their high reactivity and short lifetime.

Sigaeva et al. recently published an article [Small, 18(44), p. 2105750; DOI: 10.1002/smll.202105750] disclosing diamond-based nanoscale quantum relaxometry for sensing free radical production in cells. The contents of this article are incorporated herein by reference. As explained in the article, diamond magnetometry makes use of fluorescent defects in nanodiamonds to convert magnetic resonance signals into fluorescence. According to the summary of the experimental procedure, a nanodiamond containing NV centers is localized in or nearby a living cell and visualized through confocal microscopy. A laser pulse train is then used to pump the NV centers into a bright state. The emission of the nanodiamond particle is then recorded within a fixed time window at the beginning of the next pulse after varying dark times. The collected fluorescence intensity reveals whether the NV centers are still in this prepared state or if they have already relaxed to a darker equilibrium of states. The relaxation occurs more slowly at lower levels of magnetic noise in the environment. At higher levels of magnetic noise, the relaxation occurs faster, and the recorded fluorescence intensity after a given dark time is lower. Optionally, a microwave pulse can be added right before the optical pulse to specifically target the NV centers' spin resonance. Subtracting the T1 with microwave from the all-optical T1 allows to exclude competitive effects unrelated to the NV center spin.

The detection range over which fluorescent nanocrystals (FNC) such as nanodiamonds with NV centers are sensitive to their environment is limited, e.g., the sensitivity typically drops with the distance to the sixth power (~1/r⁶). So, to achieve proper detection it is desired to bring the FNC in close proximity with the region of interest, e.g. within 100 nm, preferably within 10 nm. However, suitable FNC may be relatively large, e.g. ≥10 nm or even ≥100 nm, up to 200 nm, or more. The relatively large size can make it difficult to deliver suitable FNC to regions of interest in biological tissue. In principle, uptake of FNC into single cells may occur spontaneously or can be facilitated by modification of the cells. However, the uptake of FNC into deeper regions of larger tissue samples, such as tissue slices or even in-vivo, remains challenging.

There thus remains a need for new methods, devices, and systems to improve the delivery of fluorescent nanocrystals, such as (relatively large) nanodiamonds having an NV center, into biological tissues.

### SUMMARY

The present disclosure provides methods, devices, and systems for delivering fluorescent nanocrystals such as nanodiamonds into a tissue. A microneedle assembly can be formed of a substrate with an array of microneedles for penetrating the tissue. Each microneedle is formed of a solid matrix material with a plurality of fluorescent nanocrystals dispersed throughout the material. For example, the substrate comprises a patch configured to apply the microneedle assembly to a tissue surface while penetrating the tissue with the array of microneedles. By penetrating the tissue with the one or more microneedles, and dissolving at least part of the matrix material forming the one or more microneedles, the fluorescent nanocrystals dispersed in the dissolved part of the matrix material can be effectively delivered into the tissue.

By forming the solid matrix material of biodegradable and/or biocompatible material the delivery can be advantageously applied into tissues with biological cells. For example, the biodegradable solid matrix material is configured to degrade inside the tissue within a certain timeframe. As will be understood, the one or more microneedles should be sufficiently strong to penetrate a certain distance into the tissue for delivering the fluorescent nanocrystals. For example, when using skin tissue, the fluorescent nanocrystals may be delivered into the epidermis, typically less than one millimeter into the skin. Also other types of biological tissue can be used, where the fluorescent nanocrystals may be delivered at any relevant depth. Further advantages may be achieved by connecting the fluorescent nanocrystals to respective ligands. For example, the ligands may remain in the tissue when the matrix material is dissolved used to target a specific biological structure in the tissue after being delivered into the tissue. Also other or further compounds can be used, e.g., as part of the microneedles or otherwise. For example, a compound such as collagenase can be used to loosen the tissue structure by increasing the cell-to-cell space, for facilitating uptake of the fluorescent nanocrystals into the tissue.

The present disclosure further provides methods, devices, and systems for measuring a tissue after delivering the fluorescent nanocrystals into the tissue as described herein. Typically, this includes measuring a fluorescent signal from the fluorescent nanocrystals in the tissue. In a measurement device, a tissue holder is configured to hold a slice of the tissue. Preferably, the tissue is bathed in a liquid medium and/or exposed to a controlled ambience and temperature for keeping cells of the tissue intact while measuring. The measurement device, e.g. microscope, may comprise or work together with a light source configured to deliver pulses of source light to the tissue. A light detector can be used to measure a fluorescent signal emitted by the fluorescent nanocrystals resulting from the pulses of source light. Based on the measured fluorescent signal, a biological parameter of the tissue can be determined, e.g. using an analyzer. When measuring on larger tissue samples, preferably a wavelength filter is configured to filter out auto-fluorescent light of the tissue slice, at least below a wavelength of 650 nm or 700 nm, preventing the auto-fluorescent light from reaching the light detector. Even though this may diminish the overall signal, the inventors find the filter may improve signal to noise ratio.

As will be appreciated, the fluorescent signal of respective fluorescent nanocrystals may depend on a local environment of the tissue proximate the respective fluorescent nanocrystals. For example, local electrical and/or magnetic fields may affect the fluorescence state of a colour center in the fluorescent nanocrystals. By measuring the fluorescent signal while applying a variable stimulus to the tissue, further insights may be gained gathered about the effect of such stimulus on the tissue. For example, the effects of UV irradiation, drugs, antioxidants, nutrients, and disease development on various types of tissue can be more studied. In particular, it can be determined how these stimuli may affect the presence and/or concentration of radicals in the tissue using the fluorescence signal from the fluorescent nanocrystals as a local probe. Also, other effects may be measured such as pH, pressure, temperature, et cetera.

The present disclosure further provides methods, devices, and systems for manufacturing a microneedle assembly, e.g., for the delivery and/or measurement of fluorescent nanocrystals into tissue, as described herein. According to one method, a concentration of fluorescent nanocrystals is dispersed into a liquid precursor of the matrix material. The liquid precursor with the fluorescent nanocrystals is cast into a mold which forms a negative of an array of microneedles. The liquid precursor is solidified in the mold to form the array of microneedles comprising the fluorescent nanocrystals dispersed throughout a solid matrix material. The array of microneedles can be removed from the mold to form (part of) the microneedle assembly.

### BRIEF DESCRIPTION OF DRAWINGS

These and other features, aspects, and advantages of the apparatus, systems and methods of the present disclosure will become better understood from the following description, appended claims, and accompanying drawing wherein:

FIGS. 1A-1B illustrate delivering fluorescent nanocrystals into a tissue;

FIGS. 2A-2C illustrate images of a microneedle assembly;

FIGS. 3A-3F illustrate manufacturing of a microneedle assembly, and insertion of manufactured microneedles in a tissue;

FIG. 4 illustrates measuring a tissue with fluorescent nanocrystals;

FIGS. 5A-5C illustrate images comparing fluorescent signals of FNC with background signals of the tissue using different wavelength filters;

FIGS. 6 and 7 illustrate the effect of collagenase on the uptake of FNC in various types of tissues and cells.

### DESCRIPTION OF EMBODIMENTS

Terminology used for describing particular embodiments is not intended to be limiting of the invention. As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features. It will be further understood that when a particular step of a method is referred to as subsequent to another step, it can directly follow said other step or one or more intermediate steps may be carried out before carrying out the particular step, unless specified otherwise. Likewise it will be understood that when a connection between structures or components is described, this connection may be established directly or through intermediate structures or components unless specified otherwise.

Fluorescence is understood as a process whereby a substance absorbs light or other electromagnetic radiation and re-emits it, typically at a longer wavelength. In some nanocrystals, e.g. color centers such as nitrogen-vacancy (NV) centers in diamond, fluorescence can occur due to a respective defect structure within the crystal lattice. For example, an NV center consists of a nitrogen atom adjacent to a vacancy, creating an electronic environment capable of absorbing and emitting photons. These and other color centers formed as nanocrystal may be beneficial for various properties such as exhibiting a stable and bright fluorescence, In other or further nanocrystals, e.g. nanoscale semiconductor particles, fluorescence can occur due to quantum confinement effects that alter the electronic and optical properties of the material. When these nanocrystals absorb photons, their electrons are excited to higher energy states. The subsequent return of these electrons to their ground state results in the emission of light, with the emission wavelength being tunable based on the size and composition of the nanocrystals.

The invention is described more fully hereinafter with reference to the accompanying drawings, in which embodiments of the invention are shown. In the drawings, the absolute and relative sizes of systems, components, layers, and regions may be exaggerated for clarity. Embodiments may be described with reference to schematic and/or cross-section illustrations of possibly idealized embodiments and intermediate structures of the invention. In the description and drawings, like numbers refer to like elements throughout. Relative terms as well as derivatives thereof should be construed to refer to the orientation as then described or as shown in the drawing under discussion. These relative terms are for convenience of description and do not require that the system be constructed or operated in a particular orientation unless stated otherwise.

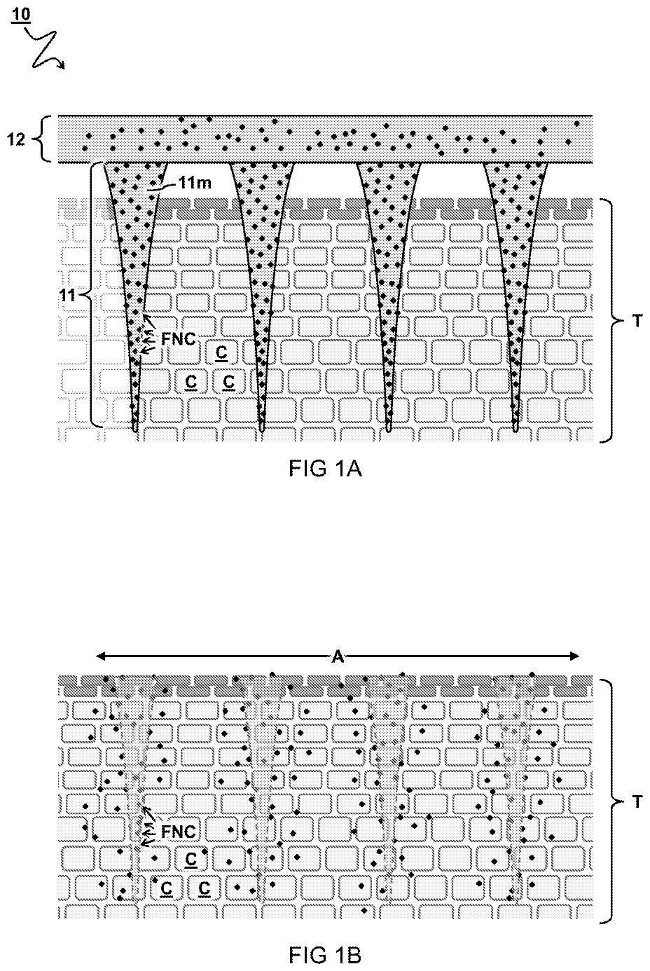

FIGS. 1A-1B illustrate delivering fluorescent nanocrystals (FNC) into a tissue “T”. In some embodiments, the FNC are delivered using one or more microneedles 11. In one embodiment, each microneedle 11 comprises a plurality of FNC. In another or further embodiment, the FNC are dispersed throughout a matrix material 11m. A matrix material will be understood as a substance or medium in which another material or phase is embedded or dispersed. It acts as a host or support for the dispersed phase. The matrix material surrounds and holds the dispersed phase. As described herein, the matrix material may hold together the FNC dispersed therein. The matrix material may provide mechanical support, stability, and typically defines the overall properties of the composite material.

Some embodiments comprise penetrating the tissue with the one or more microneedles 11. Other or further embodiments comprise dissolving or otherwise disintegrating the matrix material 11m, e.g. while the one or more microneedles 11 penetrate the tissue “T”. In this way, the FNC may be left behind into deeper layers of the tissue “T”.

In some embodiments, the tissue “T” comprises biological cells C. For example, the tissue “T” is human or animal skin tissue. Also other biological tissues can be used. In one embodiment, the microneedles 11 are configured to penetrate into an epidermis of the skin tissue for delivering the FNC in the epidermis. For example, the microneedles 11 are configured to penetrate at least 0.1 mm into the epidermis, preferably at least 0.2 mm, more preferably at least 0.5 mm, e.g. up to 1 mm, or more. So it will be understood that the matrix material 11m (with the FNC dispersed therein), is suitable to form microneedles which are capable of penetrating into (biological) tissue such as skin tissue, organ tissue, et cetera. For example, the microneedles have sufficient mechanical strength and rigidity to enable the microneedles to withstand the forces required for insertion into the tissue without bending or breaking. Accordingly, the microneedles can maintain their structural integrity during penetration. Preferably, the material exhibits a certain degree of flexibility and elasticity to accommodate the bending and deformation that may occur during penetration. This property may help to reduce the risk of microneedle breakage and/or enhance the ability to conform to irregular tissue surfaces.

Preferably, the matrix material 11m, is capable of spontaneously dissolving or otherwise disintegrating. Most preferably, this occurs when the matrix material 11m is brought in contact with the tissue when the microneedles 11 are inserted. In one embodiment, the matrix material 11m comprises or essentially consists of a biodegradable material. Biodegradable materials typically possess the ability to undergo decomposition and breakdown through biological processes, such as enzymatic or microbial action. These materials are typically composed of organic compounds that can be recognized and metabolized by biological systems. Their molecular structure may include easily cleavable chemical bonds or functional groups, facilitating enzymatic or microbial attack and subsequent degradation. Alternatively, or additionally, biodegradable materials may be dissolved or otherwise disintegrated by contact with a solvent such as water.

Preferably, the biodegradable material is also biocompatible, e.g. producing non-toxic degradation products that can be assimilated into natural biological processes. Examples of biodegradable and/or biocompatible materials suitable for microneedle fabrication may include polymers, e.g. comprising or formed of compounds including one or more of hyaluronic acid, lactic acid, glycolic acid, lactic-co-glycolic acid, caprolactone, polyvinyl alcohol, gelatin, et cetera. Alternatively, or additionally suitable matrix materials may include sugar-based materials such as dextran, sucrose, trehalose; and/or protein-based materials such as collagen, silk fibroin. Also other materials or compounds can be used.

In some embodiments, the biodegradable material is configured to (at least partially) degrade inside the tissue after insertion, e.g. within a timeframe of less than ten hours, preferably less than one hour, more preferably less than half an hour, e.g. within ten minutes, or less. On the one hand the matrix material 11m is preferably capable of degrading within the tissue “T” in a reasonable timeframe to continue an experiment. On the other hand, the microneedles are preferably sufficiently stable so as not to easily degrade before insertion. For example, degradation rate can be controlled by adjusting factors such as polymer composition, molecular weight, and processing methods. Alternatively, or in addition, the microneedles may be kept in a controlled environment to prevent degradation before insertion. In principle, it is not required that the microneedles 11 are completely disintegrated. For example, a substantial amount of FNC may be released while part of the microneedles 11 is disintegrated; and the remainder of the microneedles 11 can be retracted from the tissue “T” before complete disintegration. Preferably, at least 10% of the matrix material 11m forming the microneedles 11 penetrating the tissue is dissolved within the said timeframe, more preferably, at least 50%, up to 90% or even 100%. So, it will be understood that the microneedles 11 may be partially or completely dissolved inside the tissue “T”.

Contrary to the matrix material 11m, the FNC may be relatively stable, e.g. not biodegradable or less biodegradable than the matrix material 11m. This allows the FNC to remain in the tissue “T” for measurements, after the matrix material 11m has degraded. While the FNC may not be degradable, they are preferably non-toxic, e.g. having no or minimal damaging effect on the tissue “T” in which they are delivered. For example, fluorescent nanodiamonds may be relatively inert and/or biocompatible (non-toxic) in numerous different cell types. Biocompatible properties of the matrix material 11m and/or FNC such as nanodiamonds may allow advantageous use of the present teachings for the measurement of tissues with living cells, e.g. in-vitro and/or in-vivo. Furthermore, when the microneedles are relatively short, delivery of FNC may be limited, e.g., to the outer layers of the skin (epidermis), which is frequently renewed, thus shedding the FNC even if they do not degrade.

In some embodiments, the FNC forming part of the microneedle(s) 11 are connected to respective ligands, such as antibodies, aptamers, charged moieties, lipids, and/or molecules that are recognized by receptors, wherein the FNC connected to respective ligands remain in the tissue “T” when the matrix material 11m is dissolved, wherein the respective ligands are configured to bind to a specific biological structure after being delivered into the tissue “T”. Advantageously, this may help to further target specific biological structures after the FNC are delivered into the tissue.

In some embodiments, the microneedle 11 is part of a microneedle assembly 10 with a plurality of the microneedles 11 arranged on a substrate 12. In one embodiment, the microneedle assembly 10 is configured to deliver the FNC across an area “A” of the tissue “T”. Preferably, the microneedle assembly 10 comprises at least four microneedles, more preferably at least ten, twenty, fifty, one hundred, e.g. up to one thousand microneedles, or more. In another or further embodiment, the microneedles occupy an area of at least one square millimeter, e.g. up to one square centimeter or more. In another or further embodiment, the density of microneedles is at least ten microneedles per square centimeter, preferably at least one hundred microneedles per square centimeter, e.g. up to one thousand microneedles per square centimeter, or more. For example, a microneedle assembly may be formed as a 10×10 array of 100 microneedles on an area of 1 cm×1 cm.

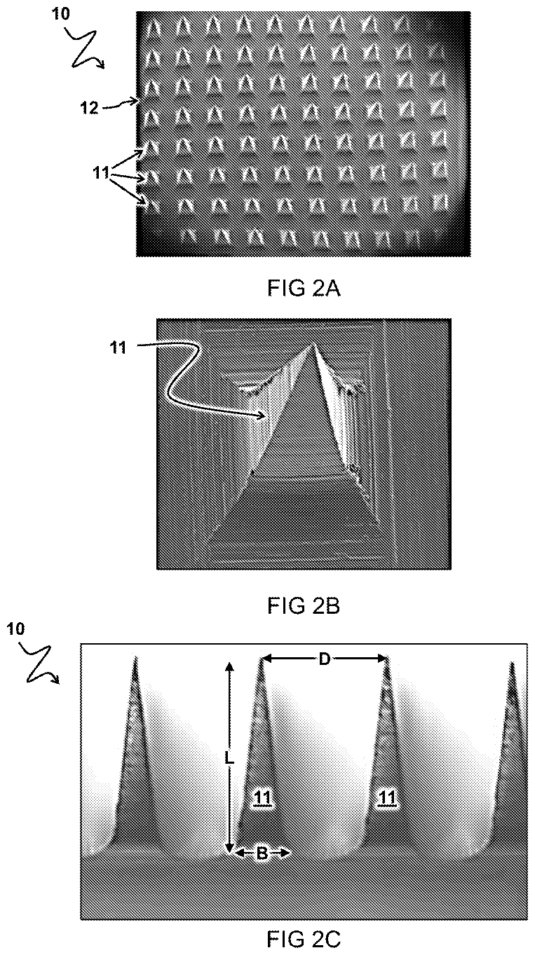

FIGS. 2A-2C illustrate images of a microneedle assembly 10, e.g.

for delivering FNC, as described here. In one embodiment, each microneedle 11 has a length “L” (transverse or perpendicular to the substrate 12) of less than one millimeter. For example, the microneedles 11 as shown have a length L of 700 μm. Also longer or shorter needles can be used, e.g. with a length between 0.1-10 mm, preferably between 0.3-5 mm, most preferably between 0.5-1.5 mm. The preferred length may depend on the tissue “T” to be penetrated. For example, when the needles are used for delivering fluorescent nanocrystals (FNC) into an epidermis, the needles may have a length similar to, or shorter than a thickness of the epidermis.

For example, the needles may have a length shorter than 1.5 mm, shorter than 1 mm, or even shorter than 0.5 mm.

Preferably, the microneedles 11 are relatively narrow and/or sharp, e.g. having a base width “B” less than a respective needle length “L” by at least a factor two, preferably at least a factor three. For example, the microneedles 11 as shown have a base width “B” of 200 μm. Also wider or narrower needles can be used. For example, narrower needles may be sharper, but less robust; and vice-versa. Preferably, adjacent microneedles 11 in the microneedle assembly 10 are relatively close, e.g. separated by a distance “D” less than a respective length L of the microneedles 11 and/or having a distance “D” less than three times the base width “B”. For example, in the image shown, the microneedles 11 are separated by a distance of 500 μm. Also smaller or larger distances can be used. The smaller the distance, the higher the density of needles and concentration of FNC which can be delivered per unit area.

Preferably, the FNC are relatively small compared to the size (L and/or B) of the microneedles 11, e.g. smaller than the length “L” and/or base width “B” of the needles by at least a factor one hundred, more preferably at least a factor one thousand. Typically the FNC have a (maximum) diameter between 1-1000 nm, preferably between 10-500 nm, most preferably between 100-250 nm. The inventors find that using relatively large FNC, e.g. >100 nm, may be beneficial in eliminate problems from autofluorescence in measurement of tissue. On the other hand, the FNC are still relatively small compared to biological cells forming the tissue, e.g. at least a factor hundred smaller than the cells. For example, the average human skin cell is about 30 μm in diameter, and the FNC are preferably <250 μm. Also other types of tissue may be used, with corresponding cells and suitable FNC having possible other sizes.

In a preferred embodiment, the FNC comprise or essentially consist of fluorescent nanodiamonds (FND). In principle, FND can be produced with various processes. Examples may include detonation nanodiamonds (DNDs) and nanodiamonds from a high pressure high temperature HPHT source. Typically, DNDs are relatively small, e.g. 5 nm, which may be less suitable for the present applications than larger nanodiamonds, e.g. produced by HPHT. Most preferably, each nanodiamond comprises at least one NV center, or multiple NV centers. In general, an NV center is a defect in the lattice of a diamond crystal formed of carbon atoms that is created when a nitrogen atom replaces a carbon atom in the diamond lattice and a vacancy missing atom is present in the lattice at the same time. So, the amount of NV centers per nanodiamond may be controlled by the concentration of nitrogen. NV centers have a unique electronic structure that gives them special properties. They have a spin triplet ground state and a spin singlet excited state, which can be optically pumped between the two states. This allows them to be used, e.g., as a sensor for magnetic fields and other physical quantities, such as pressure, temperature, and strain.

Alternatively, or in addition to NV centers, also other types of color centers can be used in nanodiamonds, e.g. silicon-vacancy centers in diamond, aluminum-vacancy centers in diamond, and divacancy centers in diamond. Alternatively, or in addition to nanodiamonds formed of crystalline carbon atoms, also other crystalline materials can be used to form nanocrystals with respective (fluorescent) color centers. Examples may include nitrogen-vacancy centers in silicon carbide, silicon vacancy centers in silicon carbide, carbon-antisite centers in silicon carbide, aluminum-vacancy centers in aluminum nitride, carbon-antisite centers in aluminum nitride, carbon-vacancy centers in aluminum nitride, and iron-vacancy centers in aluminum nitride.

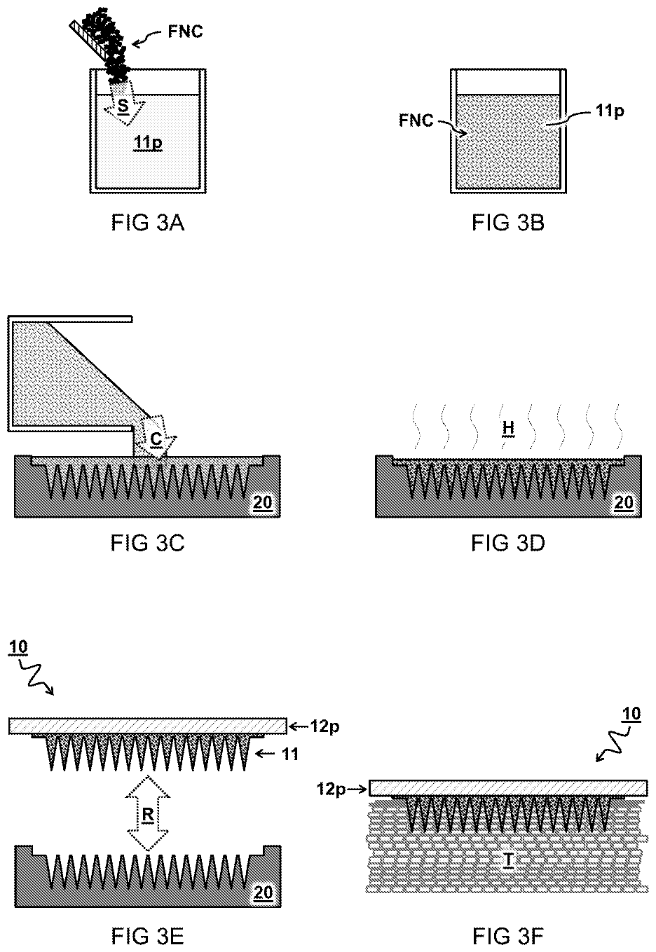

FIGS. 3A-3F illustrate manufacturing of a microneedle assembly 10, and insertion of manufactured microneedles 11 in a tissue “T”. In some embodiments, the microneedles 11 are fabricated using micro-molding techniques. For example, a master mold with the desired microneedle geometry is created using techniques like photolithography or laser ablation. The mold is then filled with a solution or gel of the matrix material precursor, which is allowed to solidify. After solidification, the microneedle assembly is demolded, resulting in individual hyaluronic acid microneedles.

One embodiment, e.g. as shown in FIGS. 3A-3B, comprises a step “S” of dispersing a concentration of FNC into a liquid precursor 11p of a matrix material 11m. Another or further embodiment, e.g. as shown in FIG. 3C, comprises a step “C” of casting a liquid precursor 11p with FNC cast into a mold 20. For example, the mold 20 forms a negative of the microneedle assembly 10. In principle, also other mold shapes may be used to manufacture any desired shape of a matrix material 11m comprising FNC. Another or further embodiment, e.g. as shown in FIG. 3D, comprises a step “H” of hardening and/or solidifying a liquid precursor 11p with FNC in a mold 20 to form a microneedle assembly 10 comprising the FNC dispersed throughout a hardened and/or solid matrix material 11m. For example, the step “H” may include evaporating of a solvent in the liquid precursor 11p and/or polymerizing of monomers to form the matrix material 11m. Also other solidifying steps and processes can be envisaged. Another or further embodiment, e.g. as shown in FIG. 3E, comprises a step “R” of removing a hardened matrix material 11m with FNC from a mold 20 to form a microneedle assembly 10.

In some embodiments, the liquid precursor of the matrix material 11m is formed by a solution comprising the matrix material 11m dissolved in a suitable solvent, to which also a concentration of FND can be added. In one exemplary implementation, an FND-loaded microneedle (MN) array (100 MN/1×1 cm²) was fabricated by solvent-casting of 10 w/v % polyvinylpyrrolidone (PVP) solution, 3% Hyaluronic acid and 10 μg/ml ultrasonicated FND solution in deionized water, following a centrifugation and degassing step in a vacuum chamber at 25° C. to fill the cavities of a negative (PDMS) mold. The FND-loaded MN patch was dried at 25° C. for 48 h, carefully peeled from the PDMS mold, and stored in a desiccator to keep it dry. In this example, the matrix material 11m and/or its precursor comprises hyaluronic acid. Hyaluronic acid is a glycosaminoglycan, which is a type of long, linear polysaccharide made up of repeating disaccharide units. The repeating units of hyaluronic acid may consist of D-glucuronic acid and N-acetyl-D-glucosamine. For example, this may form the solidified matrix material 11m.

Some aspects of the present disclosure can be embodied as microneedle assembly 10, e.g. manufactured according to the methods described herein, or otherwise. In one embodiment, the microneedle assembly 10 comprises a substrate 12 with a (one or two-dimensional) array of microneedles 11 for penetrating a tissue. In another or further embodiment, each microneedle 11 is formed of a matrix material 11m with a plurality of FNC dispersed throughout the matrix material 11m. Preferably, the matrix material 11m is biodegradable and/or biocompatible. In some embodiments, e.g. as illustrated in FIG. 3E, the substrate 12 comprises a patch 12p. In other or further embodiments, the patch 12p is configured to apply the microneedle assembly 10 to a tissue surface while penetrating the tissue “T” with the array of microneedles 11 as illustrated in FIG. 3F. For example, the patch may comprise a sticky surface to adhere the microneedle assembly 10 to a tissue surface while the matrix material 11m forming the microneedles 11 is allowed to at least partially dissolve, thereby delivering the FNC into the tissue “T”.

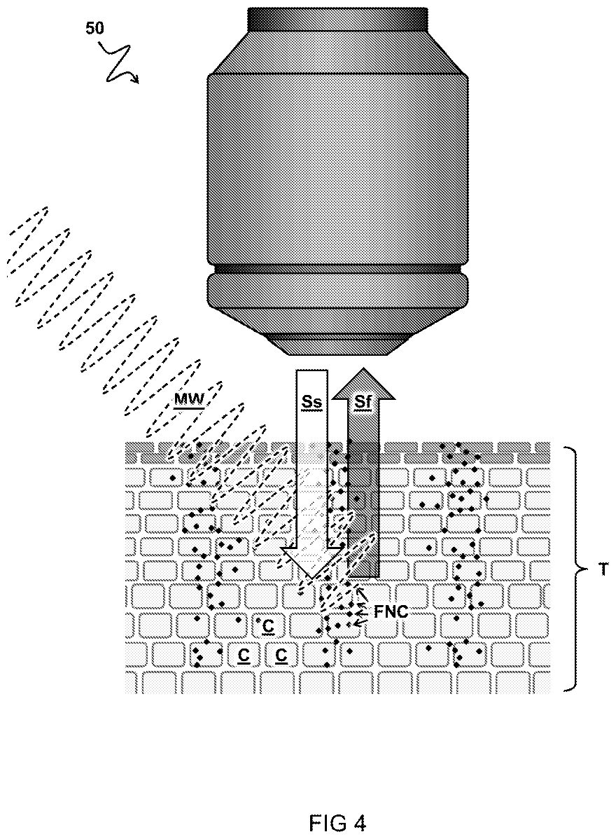

FIG. 4 illustrates measuring a tissue “T” with FNC. In some embodiments, the method comprises delivering FNC into the tissue “T” using a microneedle assembly as described herein, or otherwise. In one embodiment, a fluorescent signal “Sf” is measured from the FNC in the tissue. For example, the measured fluorescent signal “Sf” results from the FNC in the tissue “T” being excited by a pulse of source light “Ss”.

Alternatively, or in addition to applying source light “Ss”, also other or further signals may be applied to the tissue. In one embodiment, the tissue “T” is exposed to a microwave signal MW. For example, the microwave signal MW may affect the fluorescent state of the tissue “T”. In another or further embodiment, a magnetic field is applied to the tissue “T” (not illustrated here).

In some embodiments, the measured fluorescent signal “Sf” of respective FNC is dependent on, or representative of, a local environment of the tissue “T” proximate the respective FNC. In one embodiment, the measured fluorescent signal “Sf” is used to determine a presence and/or concentration of radicals in the tissue “T”. In another or further embodiment, the fluorescent signal “Sf” and/or the presence and/or concentration of radicals in the tissue “T” is measured as a function of a stimulus applied to the tissue. For example, the applied stimulus to measure radicals or other factors affecting the FNC, may include one or more of applied UV irradiation, applied drug or antioxidant, applied nutrients, certain disease development. For example, these or other stimuli may influence the oxidative state of the tissue which can be measured. In one embodiment, measurements of the fluorescent signal “Sf” are performed with and without the applied stimulus and/or applying different degrees of the stimulus (e.g. different amounts of UV radiation). In other or further embodiments, the measured fluorescent signal is used to determine one or more of a pH, pressure, temperature, and magnetic field in the tissue “T”.

The inventors find there are major differences between the measurement of FNC in cells and the measurements in larger tissues as described herein. First, the tissue is thicker and thus it might be necessary to measure deeper in the material. Second, tissues usually rely on a blood supply and do not have as much direct access to medium and are thus more fragile. Third, tissues have stronger autofluorescence than cells. Furthermore, while cells adhere to the bottom of a dish and thus stay stationary during a measurement, tissues do not and thus have to be held in place. To eliminate problems from autofluorescence the inventors prefer the use of larger nanodiamonds, e.g. 120 nm. Additionally, the inventors collected at a different wavelength. While in cells light may be typically collected above 600 nm, in tissues the inventors find it advantageous to collect at a higher wavelength above 650 nm or even above 700 nm. This way the signal may be reduced but the amount of background is reduced even more. Finally, while for most cells it may be sufficient to measure at room temperature and ambient air, the inventors find that for measuring tissues experiments should preferably be conducted at more controlled environment, e.g. +37° C. and most preferably in presence of 5% CO₂.

Some aspects of the present disclosure can be embodied as measurement device 50 configured to measure FNC delivered into a tissue “T”. In one embodiment, measurement device comprises or is otherwise configured to use a tissue holder (not shown) configured to hold a slice of the tissue “T”. Preferably, the tissue holder is configured to hold the tissue in place and allow a medium e.g. comprising nutrients to get close to the tissue. For example, to avoid stress to the tissue the inventors developed a custom-made stainless-steel holder which is reusable, easy to operate, and clean. There are several metal threads fence in the holder to confine the movement of the tissue. To avoid too much pressure, the height between threads and dish can be adjusted according to the thickness of the tissue. Optionally, there can also be one or more holes in the wall of the holder to let the medium pass into and/or out of the holder.

In some embodiments, the measurement device 50 comprises or is otherwise configured to use a light source (not shown) configured to deliver pulses of source light “Ss” to the tissue “T”. In another or further embodiment, the measurement device 50 comprises or is otherwise configured to use a light detector (not shown) configured to measure a fluorescent signal “Sf” emitted by the FNC resulting from the pulses of source light Sf. Also other sources or detectors may be included in the device e.g. sources and/or detectors for generating and/or measuring microwaves MW. Also, a source for generating a constant and/or variable magnetic field, e.g. gradient field, may be included as part of the device, or otherwise coupling with the tissue T.

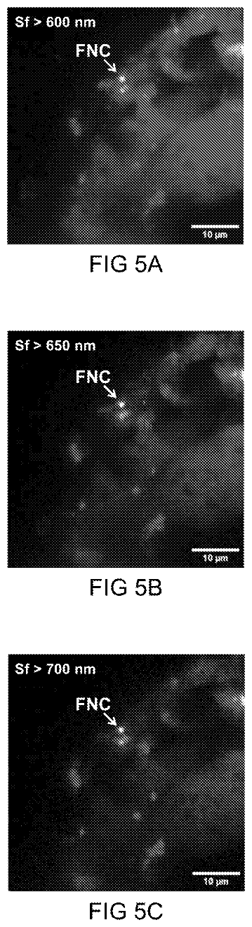

In some embodiments, the measurement device 50 comprises or is otherwise configured to use an analyzer configured to determine a biological parameter of the tissue “T” based on the measured fluorescent signal “Sf”. Preferably, the measurement device comprises a wavelength filter (not shown) configured to filter out fluorescent light below a wavelength of 650 nm, preferably below 700 nm. For example, FIGS. 5A-5C illustrate measurements with different wavelength filters, where it may be noted that the FNC signal over tissue background is improved for the wavelength filtering above 650 nm, compared to 600 nm, and further improved for the wavelength filtering above 700 nm.

In some embodiments, methods for delivering FNC comprises applying a compound such as collagenase configured to loosen the structure of the tissue “T”. This may facilitate uptake of the FNC into the tissue “T”. For example, the compound may partially break down parts of the cell walls, extracellular matrix and/or connection between cells. In one embodiment, the compound forms part of the microneedles 11, e.g. dispersed in the matrix material 11m, or otherwise forming part thereof. For example, the compound may be released together with the FNC when the matrix material 11m dissolves in the tissue “T”. In another or further embodiment, the compound may be applied before, after, or during the insertion of the microneedles 11. It can also be envisaged to apply the compound as an alternative to the microneedles 11 for delivering the FNC into a tissue. For example, FNC can be applied onto the surface of a tissue treated with the compound, or the compound may be mixed with FNC and applied to the tissue. The treatment of tissue with the compound using microneedles, or otherwise, may be most applicable to the measurement of tissue in-vitro, e.g. tissue slices.

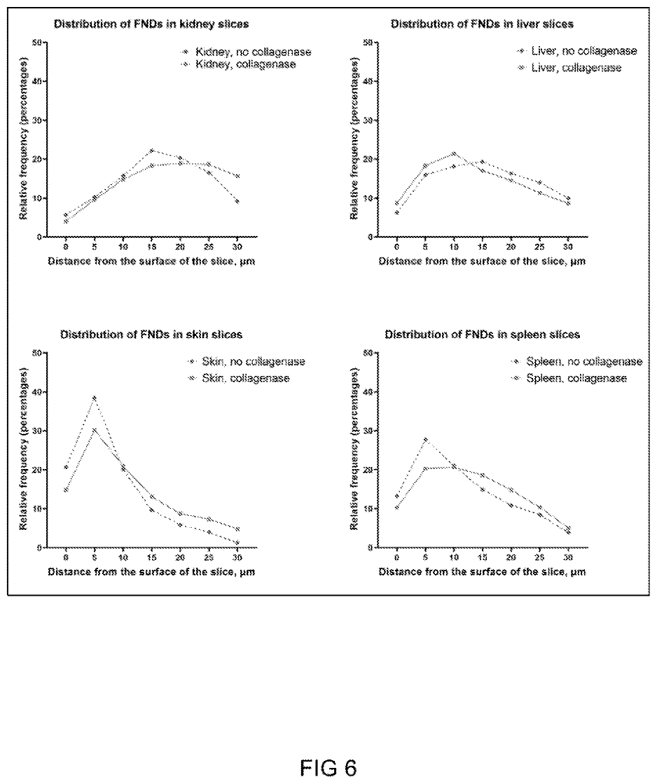

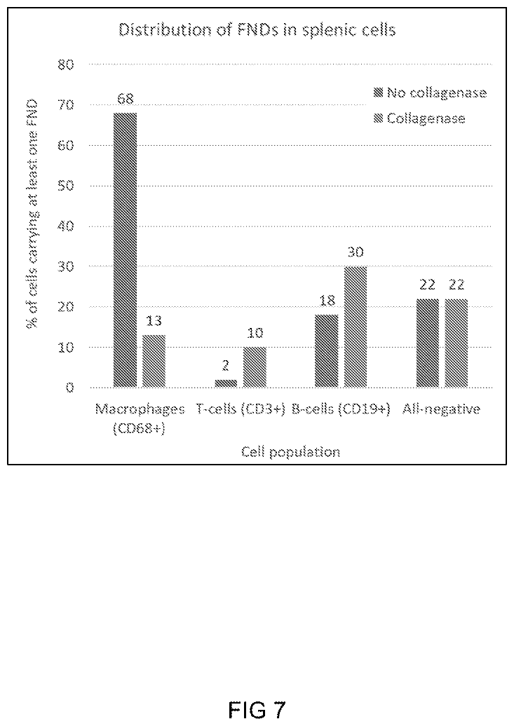

FIG. 6 illustrates various measurements of the uptake of fluorescent nanodiamonds (FNDs) in various types of tissue including kidney slices, liver slices, skin slices, and spleen slices. A comparison is shown of the uptake distribution with and without using collagenase. As will be appreciated, the uptake into deeper regions of the tissue may be improved by the use of collagenase. FIG. 7 illustrates further measurement of the uptake of FNDs in splenic cells. In particular, the figure illustrates how the percentage of different types of cells carrying at least one FND may be affected by the use of collagenase. As illustrated, the use of collagenase may promote a relative increase of the uptake of FND into other cell-types than macrophages.

For the purpose of clarity and a concise description, features are described herein as part of the same or separate embodiments, however, it will be appreciated that the scope of the invention may include embodiments having combinations of all or some of the features described. For example, while embodiments were shown for specific types of tissue under specific conditions, also alternative ways may be envisaged by those skilled in the art having the benefit of the present disclosure for achieving a similar function and result. For example, alternative to applying the microneedle assembly to a skin tissue, the assembly or other methods for delivering FNC could be using for instance tissue blocks, full organs, or even living or dead organisms. The various elements of the embodiments as discussed and shown offer certain advantages, such as improving the uptake of FNC in a relatively non-invasive way and/or for tissues or cells which normally would not take up FNC at all. Of course, it is to be appreciated that any one of the above embodiments or processes may be combined with one or more other embodiments or processes to provide even further improvements in finding and matching designs and advantages. It is appreciated that this disclosure offers particular advantages, for delivering fluorescent nanodiamonds to a skin tissue, in particular the epidermis, and in general can be applied for any application where FNC or similar particles need to enter any type of tissue.

In interpreting the appended claims, it should be understood that the word “comprising” does not exclude the presence of other elements or acts than those listed in a given claim; the word “a” or “an” preceding an element does not exclude the presence of a plurality of such elements; any reference signs in the claims do not limit their scope; several “means” may be represented by the same or different item(s) or implemented structure or function; any of the disclosed devices or portions thereof may be combined together or separated into further portions unless specifically stated otherwise. Where one claim refers to another claim, this may indicate synergetic advantage achieved by the combination of their respective features. But the mere fact that certain measures are recited in mutually different claims does not indicate that a combination of these measures cannot also be used to advantage. The present embodiments may thus include all working combinations of the claims wherein each claim can in principle refer to any preceding claim unless clearly excluded by context.

1. A method comprising:
providing one or more microneedles, wherein each microneedle comprises a plurality of fluorescent nanocrystals dispersed throughout a solid matrix material;
penetrating a tissue with the one or more microneedles; and
dissolving at least part of the matrix material forming the one or more microneedles penetrating the tissue, thereby delivering the fluorescent nanocrystals dispersed in the dissolved part of the matrix material into the tissue.

2. The method according to claim 1, wherein the tissue comprises biological cells, and the solid matrix material is a biodegradable material, wherein the biodegradable solid matrix material forming the one or more microneedles penetrating the tissue is configured to degrade inside the tissue within a timeframe of less than one hour.

3. The method according to claim 1, wherein the tissue is skin tissue, wherein the one or more microneedles are configured to penetrate at least one tenth of a millimeter into an epidermis of the skin tissue for delivering the fluorescent nanocrystals into the epidermis.

4. The method according to claim 1, wherein the microneedles are part of a microneedle assembly comprising an array of the microneedles arranged on a substrate,
wherein each microneedle has a maximum needle length, extending transverse to the substrate, of less than one millimeter;
wherein the microneedles have a maximum base width, parallel to the substrate, which is less than the needle length by at least a factor three;
wherein adjacent microneedles in the microneedle assembly are separated by a distance less than the needle length.

5. The method according to claim 1, wherein the fluorescent nanocrystals comprise fluorescent nanodiamonds with an average diameter between 100-250 nm, wherein each nanodiamond comprises at least one nitrogen-vacancy center.

6. The method according to claim 1, wherein the fluorescent nanocrystals forming part of the one or more microneedles are connected to respective ligands, wherein the fluorescent nanocrystals connected to respective ligands remain in the tissue when the matrix material is dissolved, wherein the respective ligands are configured to bind to a specific biological structure in the tissue after being delivered into the tissue.

7. The method according to claim 1, comprising applying a compound configured to loosen a cellular structure of the tissue, for facilitating uptake of the fluorescent nanocrystals into the tissue.

8. The method according claim 1, wherein the matrix material and the fluorescent nanocrystals are biocompatible and non-toxic.

9. The method according to claim 1, wherein the tissue is skin and said penetrating of the tissue with the one or more microneedles is limited to an epidermis of the skin.

10. The method according to claim 1, wherein the tissue is a tissue slice and the method is performed in-vitro.

11. The method according to claim 1, further comprising:
measuring a fluorescent signal from the fluorescent nanocrystals in the tissue.

12. The method according to claim 11, wherein the measured fluorescent signal of respective fluorescent nanocrystals is dependent on a local environment of the tissue proximate the respective fluorescent nanocrystals.

13. The method according to claim 12, wherein the fluorescent signal is measured as a function of a variable stimulus applied to the tissue, wherein the variable stimulus includes one or more of an amount UV irradiation applied to the tissue, an amount of a drug or antioxidants applied to the tissue, an amount and/or type of nutrients applied to the tissue, and a disease development in the tissue.

14. The method according to claim 13, wherein the measured fluorescent signal is used to determine a presence and/or concentration of radicals in the tissue as function of the variable stimulus.

15. A method of manufacturing a microneedle assembly, the method comprising:
dispersing a concentration of fluorescent nanocrystals into a liquid precursor of a matrix material;
casting the liquid precursor with the fluorescent nanocrystals into a mold which forms a negative of an array of microneedles;
solidifying the liquid precursor in the mold to form the array of microneedles comprising the fluorescent nanocrystals dispersed throughout a solid matrix material; and
removing the array of microneedles from the mold forming part of the microneedle assembly.

16. The method according to claim 15, wherein the fluorescent nanocrystals comprise fluorescent color centers.

17. A microneedle assembly comprising a substrate with an array of microneedles for penetrating a tissue, wherein each microneedle is formed of a biodegradable solid matrix material with a plurality of fluorescent nanocrystals dispersed throughout the solid matrix material.

18. The microneedle assembly according to claim 17, wherein the fluorescent nanocrystals comprise fluorescent color centers.

19. The method according to claim 11, wherein measuring the fluorescent signal comprises:
holding a slice of the tissue bathed in a liquid medium and exposed to a controlled ambience and temperature for keeping cells of the tissue intact;
delivering pulses of source light to the tissue; and
determining a biological parameter of the tissue based on the measured fluorescent signal.

20. The method according to claim 19, further comprising filtering out auto-fluorescent light of the tissue slice, at least below a wavelength of 650 nm, preventing the auto-fluorescent light from being measured.
